(19) United States
(12) Patent Application Publication
(10) Pub. No.: US 2014/0309727 A1
(43) Pub. Date: Oct. 16, 2014

(54) MITRAL HEART VALVE PROSTHESIS AND ASSOCIATED DELIVERY CATHETER

(71) Applicant: ST. GEORGE MEDICAL, INC. (BVI), Freeport (BS)

(72) Inventors: JOSEPH LAMELAS, MIAMI BEACH, FL (US); EMMANUEL GAILLARD, MARSEILLE CEDEX 9 (FR)

(21) Appl. No.: 14/073,043

(22) Filed: Nov. 6, 2013

(30) Foreign Application Priority Data
Apr. 12, 2013 (FR) 1353362

(51) Int. Cl. A61F 2/24

(52) U.S. Cl. CPC A61F 2/2427
USPC 623/2.11

(57) ABSTRACT

The invention relates to a mitral heart valve prosthesis and a delivery catheter to carry and deploy such a prosthesis. The invention allows to effectively treat a pathology related to moderate to severe mitral regurgitation. Such a prosthesis implantable by catheterism includes mainly a docking station and a leaflet cooperating with the docking station. The leaflet is advantageously arranged in a configuration close to a posterior leaflet of a native mitral valve of a patient.

### CROSS REFERENCE TO RELATED APPLICATIONS

This application claims priority to French Application No. 1353362, filed Apr. 12, 2013, the contents of such application being incorporated by reference herein.

### FIELD OF THE INVENTION

The invention relates to a mitral heart valve prosthesis implantable by catheterism allowing to replace the posterior leaflet of a mitral valve and thus to treat a major cardiac valvular pathology related to moderate to severe mitral regurgitation.

### BACKGROUND OF THE INVENTION

As shown in FIG. 1, the mitral valve MV sits between the left atrium LA and the left ventricle LV of a human heart. The mitral valve apparatus consists of an annulus, two leaflets 1 and 2, tendinous chords 7 fixed respectively on one hand to said leaflets and on the other hand to the left ventricle LV through papillary muscles 7a. A normal mitral valve has two leaflets: an anterior leaflet 1 and a posterior leaflet 2. From an atrial view, the mitral annulus can be recognized as roughly elliptical line where the leaflets 1 and 2 are anchored to the atrioventricular junction in a D-shaped configuration as described in FIG. 1A. When closing the mitral valve, the two leaflets 1 and 2 are in close contact forming a single zone of apposition. The chords 7 are fibrous string-like structures connecting the ventricular face of the leaflets and the papillary muscles 7a.

Mitral valve regurgitation is a dysfunction of the mitral valve causing a blood backflow from the left ventricle LV into the left atrium LA during systole (expulsion phase of blood from the left ventricle LV into the aorta A). While trivial mitral regurgitation is frequent in healthy subjects, significant (i.e. moderate to severe) mitral regurgitation constitutes the second most prevalent valve disease after aortic heart valve stenosis. Over four million Europeans and a similar number of Americans suffer from significant mitral regurgitation. Approximately two hundred fifty thousand new patients are diagnosed with the disease annually. The disorder generally evolves insidiously over many years because the heart compensates for the regurgitant volume by left atrial enlargement, left ventricular volume overload, and progressive left ventricle dilatation. Older patients (over 50 years) with severe organic mitral regurgitation have 6% annual mortality (as compared to with 3% of mortality for moderate mitral regurgitation).

The most common causes of mitral regurgitation include ischemic heart diseases, non-ischemic heart diseases and valve degeneration. Both ischemic (coronary artery diseases) and non-ischemic (idiopathic dilated cardiomyopathy for example) heart diseases cause functional mitral regurgitation through various mechanisms, including impaired left ventricle wall motion, left ventricle dilatation, and papillary muscle displacement and dysfunction. In contrast, degenerative (or organic) mitral regurgitation is caused by structural abnormalities of the leaflets 1 and 2 of a mitral valve MV and the subvalvular apparatus, including stretching or rupture of tendinous chords.

Currently open heart surgical repair and replacement of the mitral valve are the two main options to treat mitral regurgitation. Open chest mitral valve replacement has been used to treat patients with mitral valve regurgitation since the 1960's. The patient's diseased mitral valve is replaced by either a mechanical or bioprosthetic valve. Open heart surgical procedure needs surgical opening of the thorax, the initiation of extra-corporeal circulation with a heart-lung machine, stopping and opening the heart, excision and replacement of the diseased valve, and restarting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

More recently, mitral valve repair has demonstrated advantages in terms of mortality and morbidity over replacement. This approach includes an array of valvular, subvalvular, and annular procedures aiming to restore leaflet coaptation, i.e. a normal valvular function.

However, many older patients with severe mitral regurgitation are too high operative risk. Such a surgical treatment is thus not suitable for such patients. As an example, mortality after surgical treatment for mitral valve replacement can exceed 20% for people aged over 75 years operated in less experienced centers. It is the same for patients also presenting a coronary artery disease. These “inoperable” patients thus open the way to new intervention techniques.

To reduce the mortality and morbidity of patients, less invasive transcatheter mitral valve repair or replacement approaches have been implemented in the late 1990's. Some are significantly exploited without any concrete results.

We can quote as an example and in a not exhaustive way the following used techniques:
Coronary sinus approach: Monarch System (Edwards Lifesciences), Carillion Contour Mitral System (Cardiac Dimensions), PTMA (Viacor).
Edge-to-edge repair (from Alfieri's technique): MitraClip (Abbott), Mobius (Edwards Lifesciences).
Annuloplasty: MPAS (Mitralign), Accucinch (GDS), Kardium Cinch (Kardium), QuantumCor (QuantumCor), ReCor (ReCor Medical).
Chordal replacement: DS 1000 (Neochord), Mobius II (Edwards Lifesciences), V-chordal Adjustable System (Valtech Cardio).

Current techniques of transcatheter mitral valve repair still have a high percentage of procedural failures or complications. Their long-term efficiency is relatively low in particular because of a high rate of recurrent mitral regurgitation. The acknowledgement of transcatheter mitral valve repair limits rekindled interest in transcatheter mitral valve replacement to treat mitral valve regurgitation. However, transcatheter mitral valve replacement is particularly demanding technically, more than transcatheter aortic valve replacement which was the subject of intense investigation. Transcatheter mitral valve replacement thus raises many challenges, mainly related to: the complex mitral valve and subvalvular anatomy, the absence well-structured implant site, the often multifactorial coinciding etiologies in mitral valve diseases, and the frequent occurrence of mitral valve annulus prolapse. Low attention is therefore given to transcatheter mitral valve replacement. Consequently and despite a particularly invasive side, surgical repair is the treatment usually recommended for diseases of the mitral valve.

### SUMMARY OF THE INVENTION

The invention can meet the majority of the disadvantages raised by known techniques and the challenges mentioned above. The invention consists mainly in providing a prosthetic mitral heart valve to replace or supplement the native posterior leaflet 1 of a mitral valve MV described in connection with FIGS. 1 and 1A. A new posterior leaflet which the membrane is made from biological tissues or synthetic materials is thus implanted. This leaflet cooperates with a docking station, for example in the advantageous form of a substantially tubular stent (i.e. a metal mesh) in the shape of a half-cylinder (or which the section orthogonal to the axis of revolution describes a “C”) extending in the direction of the left ventricle. Such a stent may be deployed automatically (this is called self-expanding stent) or using a balloon on which is fixed or set said stent. An optional sealing membrane and encircling the stent advantageously allows to prevent paravalvular leakage. Such a mitral valve prosthesis can be anchored during implantation in the mitral annulus using various anchoring means co-operating with the docking station.

Among the many advantages of the invention, we can mention that the invention:
the use of a technique of transcatheter mitral valve replacement significantly less invasive than open-heart surgery;
to preserve the anatomy of a mitral valve (in particular its two original leaflets) by implanting a transcatheter artificial posterior leaflet supplying the deficient native posterior leaflet while keeping the native anterior leaflet of this one;
to prevent any interaction between the native posterior leaflet of the mitral valve and the new posterior leaflet of the mitral valvular prosthesis by sticking said native leaflet against the inner wall of the left ventricle by the docking station of the prosthesis;
to maintain free the left atrium after prosthesis implantation thanks to the low profile presented by the latter;
to prevent any blocking of the left ventricular outflow tract thanks to the conservation of the native anterior leaflet and a particularly clever use of the docking station.

To this end, the invention relates to a transcatheter mitral valve prosthesis. To minimize the invasiveness resulting from its implantation in the heart of a patient and thus preserve the original anatomy, such a prosthesis includes a docking station and a leaflet comprising a membrane, said leaflet cooperating with said docking station by attachment means and being arranged in a configuration close to a posterior leaflet of a native mitral valve.

The membrane of the prosthesis leaflet may be made from one or more biological or synthetic materials.

In a first embodiment, said prosthesis leaflet is substantially flat and the attachment means compel the proximal part and the sides of said leaflet united of the docking station.

In a second embodiment, said prosthesis leaflet is substantially flat and the attachment means compel the proximal part and partially the sides of said leaflet united of the docking station, the distal part of said leaflet remaining free.

According to this second embodiment, to ensure a proper systolic coaptation of the prosthesis leaflet and the anterior leaflet of said native mitral valve, the prosthesis may further include fastening means firstly cooperating with the free part of the leaflet and secondly with the docking station. Alternatively, said fastening means may advantageously cooperate firstly with the free part of the leaflet and on the other hand, after implantation of the prosthesis within the native mitral valve of a patient, with a fibrous head of native papillary muscles or with the left ventricle of the heart of the patient. Such fastening means may comprise one or more cords preferably made of xenograft of animal pericardium coated with glutaraldehyde or in one or more synthetic cords.

In a third embodiment, the prosthesis leaflet may be made from a shape memory material. Just as in the first embodiment, the use of fastening means to optimize systolic coaptation of the prosthesis leaflet and the anterior leaflet of the native mitral valve becomes useless.

To make secured to the docking station the prosthesis leaflet according to the invention, the fastening means may consist of stitches, eyelets, staples or clips.

So that the implantation of a prosthesis according to the invention is relevant and sustainable at the ring of a native mitral valve, prosthesis may include anchoring means for anchoring of the prosthesis after implantation. Such anchoring means may consist of hooks, clamps or spikes.

According to a preferred embodiment, the docking station of a prosthesis according to the invention comprises a substantially tubular stent which shape is substantially that of a half-cylinder and wherein the proximal part of the leaflet fits into said stent in a substantially orthogonal plane to the axis of revolution of the stent. Such a stent may consist of a metal mesh of one or more wires of Nickel-Titanium or Nitinol, stainless steel, chrome-cobalt, or titanium.

To prevent paravalvular leakage, the docking station of a prosthesis according to the invention may comprise an external sealing membrane. This one can be made from animal pericardium or synthetic materials.

To implant such a prosthesis, the invention concerns, according to a second object, a delivery catheter containing a mitral valve prosthesis in accordance with the invention.

### BRIEF DESCRIPTION OF THE DRAWINGS

Other features and advantages will become more apparent upon reading the following description and on examination of the attached figures including:

FIG. 1 (already described) shows an anatomical cut of a human heart;

FIG. 1A (already described) shows the simplified anatomy of a mitral valve seen from the left atrium of a human heart;

FIG. 2 depicts a preferred embodiment of a prosthesis according to the invention;

FIGS. 3A and 3B, each show a variant of a first embodiment of a prosthesis according to the invention;

FIGS. 3C to 3E, each show a variant of a second embodiment of a prosthesis according to the invention;

FIGS. 4A to 4C describe three examples of embodiment of docking station of a prosthesis according to the invention in the form of a stent containing anchoring means;

FIGS. 5A to 5C respectively describe three steps to implant using a catheter a prosthesis according to the invention via an apical approach;

FIGS. 6A to 6D respectively describe four steps to implant using a catheter a prosthesis according to the invention via the right superior pulmonary vein.

### DETAILED DESCRIPTION OF THE INVENTION

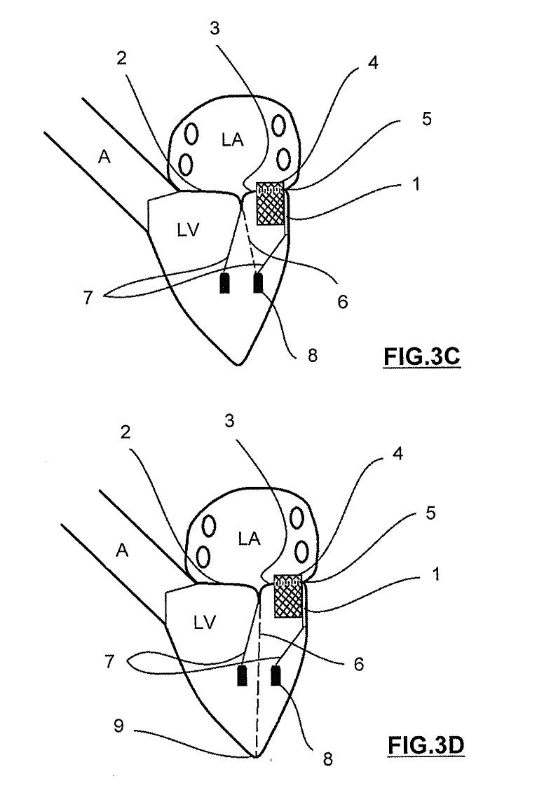

FIG. 2 shows a preferred embodiment of a mitral valve prosthesis according to the invention. Such a prosthesis allows to replace using a catheter the native posterior leaflet 1 of a mitral valve, to keep the native anterior leaflet 2 of this one and thus to treat a pathology due to mitral regurgitation.

The example of the prosthesis described in FIG. 2 includes a docking station 4 in the form of a stent or a metal mesh made of one or more wires of Nickel-Titanium or Nitinol, stainless steel, chrome-cobalt, or titanium. The configuration of the stent 4 is preferably substantially tubular, in shape of a half-cylinder which the section orthogonal to the axis of revolution creates a “C”. According to this example, the stent 4 is self-expanding or expandable using a balloon.

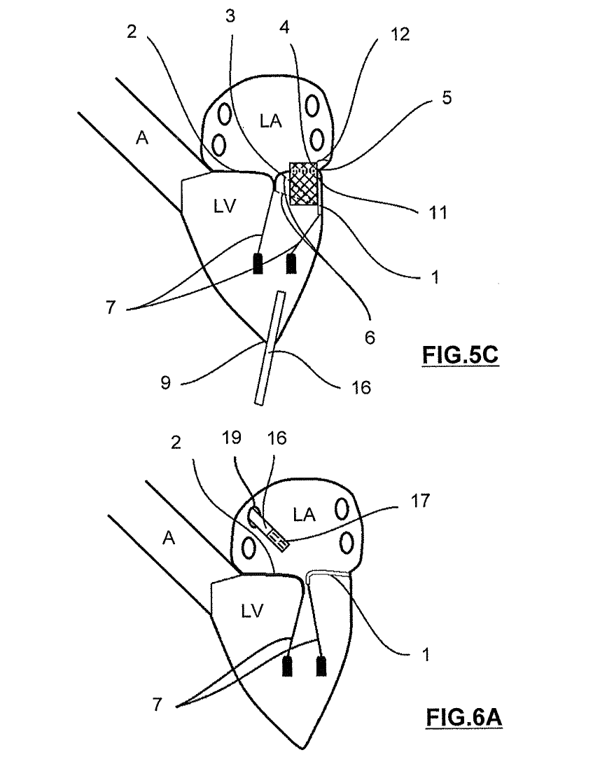

We will also see, in particular in connection with FIGS. 4A and 4B, that a mitral valve prosthesis according to the invention may be anchored to the annulus of the mitral valve using anchoring means not shown on FIG. 2 and interacting with the docking station.

The prosthesis further includes a leaflet 3 which is a membrane made for example from tissues from xenograft or standard biological materials, such as chemically or cryogenically stabilized tissues from an animal pericardium (bovine pericardium, ovine pericardium, porcine pericardium, equine pericardium). The membrane may alternatively be made from tissues from porcine cardiac valves. Synthetic materials may also be used to manufacture the membrane of the leaflet 3 of the mitral valve prosthesis: for example materials formed from a reinforced matrix of fibers such as polyurethane or polytetrafluoroethylene (PTFE).

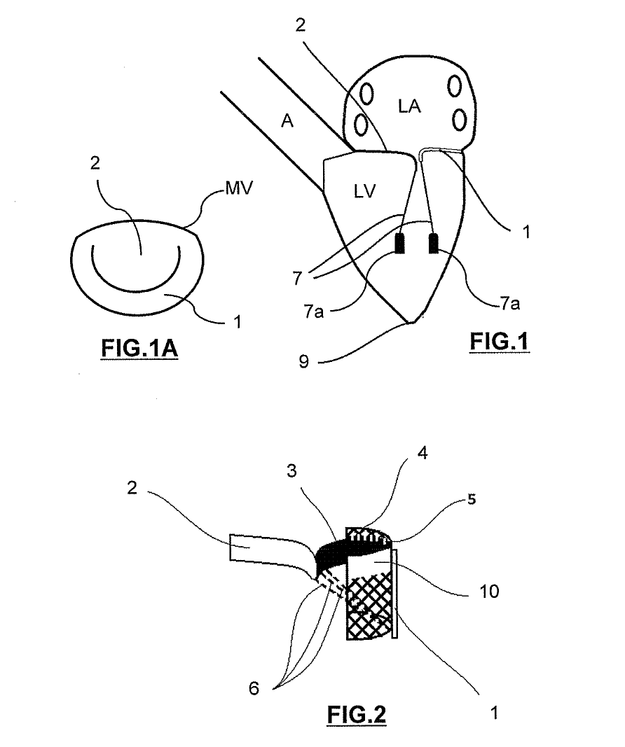

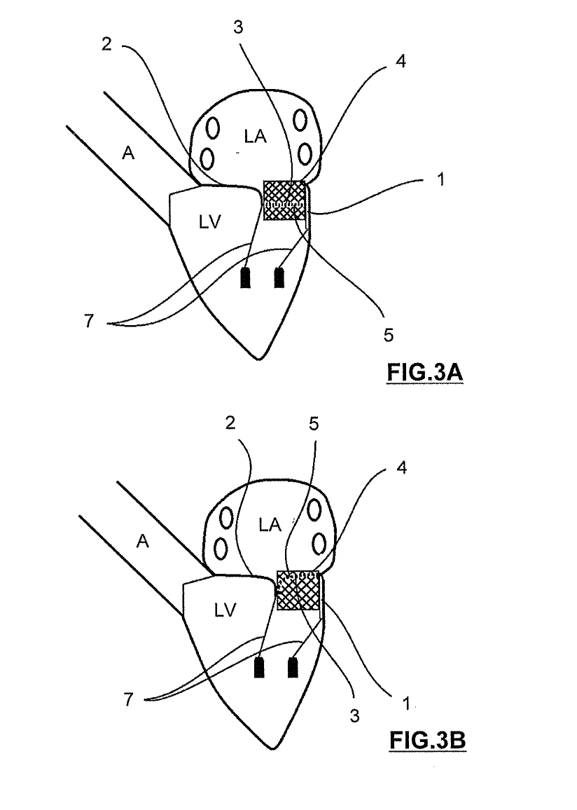

According to the invention, the leaflet 3 is arranged in a configuration close to a native posterior leaflet of a mitral valve of a patient. Such a leaflet would have a configuration close to the leaflet 1 of the native mitral valve MV described in connection with FIGS. 1 and 1A. The length of the leaflet 3 may advantageously be adjustable and adjusted to get an optimal coaptation of said leaflet 3 and the native anterior leaflet 2 of the mitral valve.

The leaflet 3 cooperates with the docking station 4. According to the example shown in FIG. 2, the proximal part of the leaflet fits into the stent 4 in a substantially orthogonal plane to the axis of revolution of the stent. The contour (i.e. the proximal end and partially the sides) of the leaflet 3 in contact with the stent 4 is secured to said stent by attachment means 5. The leaflet 3 may be directly sewn on the stent 4 using sutures. Alternatively, the stent 4 includes four eyelets allowing to fix the sutures used to attach the leaflet 3 to the stent 4. We may also use staples, clips, etc. According to the example described in connection with FIG. 2, the leaflet 3 cooperates with the stent 4 in a substantially identical plane to the one previously taken by the native posterior leaflet of the mitral valve before implantation of the prosthesis. In this regard, said plane will substantially be the one of the native anterior leaflet of the mitral valve after implantation of the prosthesis.

The leaflet 3 of a mitral valve prosthesis according to the invention may optionally be exchanged. A new leaflet may replace its predecessor if this one shows signs of wear—or just as a precaution. This increases the longevity of the prosthesis. The docking station (for example a stent) may remain as permanent, attached to the annulus of the mitral valve by its anchoring means.

According to the example described in conjunction with FIG. 2, the prosthesis further includes a sealing membrane cooperating with the stent 4 to prevent paravalvular leakage. The sealing membrane can be attached to the stent 4 using different fastening means such as staples, sutures, clips, etc. Such a membrane 10 may advantageously be made from animals pericardium (bovin, ovin, porcin or equin) or from synthetic materials such as polyester or polytetrafluoroethylene (PTFE).

FIG. 2 describes a prosthesis for which the distal part of the leaflet 3 remains free. To get a proper systolic coaptation of said leaflet 3 with a native anterior leaflet, the prosthesis preferably further comprises holding means 6, for example in the form of one or more cords, cooperating with the distal part of the leaflet 3 and the docking station 10, more precisely the lower part of the stent 4. Such holding means ensure proper closure of the mitral valve and thereby prevent any mitral regurgitation.

Cords 6 may be made from xenograft of coated glutaraldehyde pericardium (bovine, ovine, porcine and equine pericardium) and/or from synthetic materials such as polyester or expanded polytetrafluoroethylene (ePTFE).

The invention provides other configurations of prosthesis according to which it is not necessary to have recourse to such holding means 6.

As shown in FIG. 2, after implantation of the prosthesis in a human heart, the docking station sticks the native posterior leaflet 1 of the mitral valve on the inner wall of the left ventricle. This allows not to alter the anatomy of the treated heart, minimizes the risk of complications and prevents any risk of blocking the left ventricular outflow tract.

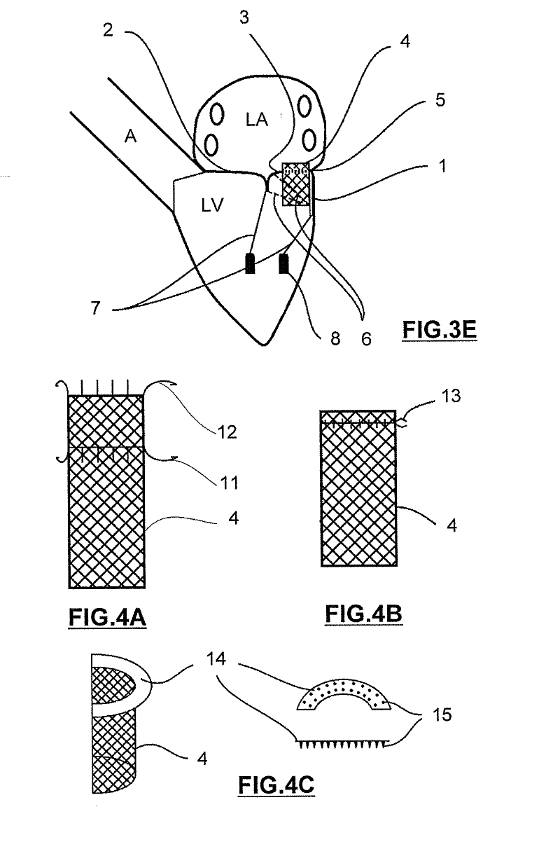

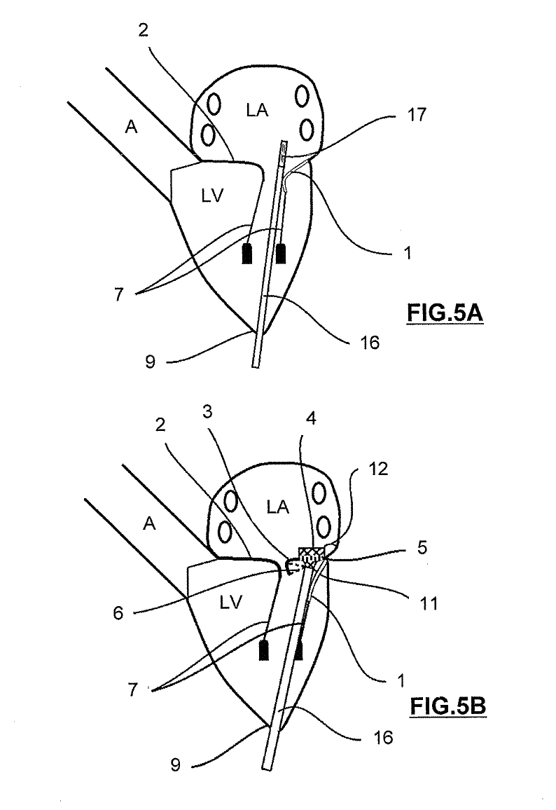

FIGS. 3A and 3B respectively describe two alternative embodiments (compared to that previously described in connection with FIG. 2) of a mitral valve prosthesis according to the invention for which the leaflet 3 of the mitral valve prosthesis does not have any free part.

The leaflet 3 is kept attached to a docking station, such as a stent, by various means of attachment such as stitches, staples, etc. The sides of said leaflet 3 and the proximal part in contact with the stent are thus joined together with said stent. After implantation of the prosthesis, as shown in FIGS. 3A and 3B, the leaflet 3 of the mitral valve prosthesis remains stationary. This one acts as a door stop. In this configuration, the prosthesis has no biological or synthetic cords 6 attached to the leaflet 3 unlike a mitral valve prosthesis as described in connection with FIG. 2. As shown in FIG. 3A, the leaflet 3 of the mitral valve prosthesis may be plan and fixed at the bottom half of the stent 4. After implantation of the prosthesis at the level of the mitral annulus, the leaflet 3 is positioned in the left ventricle, beneath said annulus, the native anterior leaflet leaning on the leaflet 3 during the closing of the mitral valve thus repaired.

The distal part of the leaflet 3 may alternatively be curved to simulate as much as possible the shape of the posterior leaflet 1 of the mitral valve. It may then be positioned and fixed to the docking station 4 at the level of the upper part of this one as shown for example in FIG. 3B.

In both cases, the original anatomy of the mitral valve is preserved. The native anterior 2 and posterior 1 leaflets are kept, connected to the papillary muscles by their native cords 7 respectively. The posterior leaflet 1 is however sticked on the inner wall of the left ventricle LV by the stent of the prosthesis 4.

FIGS. 3C to 3E respectively describe variants of arrangement according to a second embodiment of a prosthesis according to the invention.

According to this embodiment, and like the embodiment previously described in connection with FIG. 2, only a part of the leaflet 3 of a mitral valve prosthesis is attached to the stent 4. This is the proximal part (the base and partially the sides) of the leaflet. The distal part (preferably around a third of the leaflet) is left free. For optimum valve closure (occlusion conducted jointly by the leaflet 3 and the anterior leaflet 2 of the native mitral valve, the free part (distal) of the leaflet 3 cooperates with one or more biological or synthetic cords 6.

Said cords 6 are on one hand attached to the “free” distal part of the leaflet 3 and on the other hand secured to an element located downstream of the leaflet so as to exert a restoring force substantially in the direction of the apex the left ventricle LV.

As shown in FIG. 3E, said element is the lower part of the stent 4. FIG. 3E describes a prosthesis according to that already described in connection with FIG. 2 after implantation at the level of the annulus of a native mitral valve. Cords 6 (shown in dotted-lines) are thus connected to the lower base of the stent 4. One of them is attached to the end of the distal part of the leaflet 3. A second one is attached to one side of the leaflet. A third—not shown in FIG. 3E—is attached to the opposite side. The native posterior leaflet 1 is sticked on the inner wall of the left ventricle LV and keeps its native cord 7 connected to a papillary muscle 8. Similarly, the native anterior leaflet 2 remains unchanged. To fully stick the native posterior leaflet 1 on the inner wall of the left ventricle, the major part of the stent is positioned within said left ventricle LV and thus constrains said leaflet. Only the upper part of the stent 4 (for example in the form of a vertical half-cylinder) emerges from the mitral annulus in the left atrium LA. The proximal part of the leaflet 3 attached to the stent 4 is fixed in a plane substantially orthogonal to the axis of revolution of the stent 4, at the level of the upper part of the stent. The leaflet 3 may thus be substantially positioned in the same plane as the native anterior leaflet 2 of the mitral valve facing it.

In this embodiment, the stent 4 provides four main functions. Firstly, it is used as a support or docking station of the leaflet 3 of the prosthesis. It also allows to stick the native posterior leaflet 1 on the inner wall of the left ventricle LV thus avoiding any interaction between said native posterior leaflet 1 and the leaflet 3 of the mitral valve prosthesis. In addition, the stent 4 is used as anchorage point for biological or synthetic cords 6 providing a proper closure of the leaflet 3 without forcing the opening of the mitral valve. The stent is then the support of a sealing membrane (not shown in FIG. 3E but already described in connection with FIG. 2) to prevent paravalvular leakage.

Alternatively, and as presented in FIG. 3C, one or more cords 6 can be attached—either to the stent 4 but—to the fibrous head of the native papillary muscles 8 already naturally used as anchoring base of the native tendinous cords 7 of the posterior leaflet of the native mitral valve. Said leaflet 1 is also sticked by the stent 4 on the inner wall of the left ventricle LV.

In a second variant, one or more of said cords 6 may be fixed on one hand to the distal part of the leaflet 3 and on the other hand to the apex 9 of the left ventricle LV, as described in FIG. 3D.

According to a third embodiment not shown graphically, the membrane of a leaflet of a mitral valve prosthesis according to the invention may be made using a shape memory material. The closure of the leaflet of the prosthesis against the native anterior leaflet of the mitral valve is exercised by the memory shape of the membrane of the leaflet of the prosthesis. It is not necessary to use cords—such cords 6 described in connection with FIGS. 2, 3C to 3E—to exert a sufficient restoring force to prevent mitral regurgitation.

FIGS. 4A and 4B describe embodiments of anchoring means cooperating with the docking station of a prosthesis according to the invention. These means allow to fix or anchor the prosthesis during its implantation on the annulus of a native mitral valve.

According to the example described in connection with FIG. 4A, a stent 4 of a mitral valve prosthesis according to the invention may be attached to the annulus of a native mitral valve via anchoring means in the hook-shaped (or spikes) 11 which distal parts advantageously comprise harpoons to penetrate tissues of a native left ventricle. In this example, the anchoring means also comprise a second set of hooks 12 provided to penetrate the tissues of a native left atrium. Said anchoring means 11 and 12 thus provide excellent anchoring of the prosthesis at the level of the annulus of a mitral valve. The anchoring of the prosthesis (from the hooks 11 and 12) is automatically done during the deployment of the stent 4 if this one is self-expanding. It may alternatively be achieved using a balloon during the deployment of said stent via said balloon.

In a second embodiment, the anchoring means may include—as shown in FIG. 4B—a set of protruding clamps 13 respectively distributed in the periphery of the outer wall of the stent 4 at the upper part of said stent. Clamps allow to anchor the prosthesis to the annulus of a native mitral valve.

According to a third embodiment in conjunction with the FIG. 4C, the anchoring means may consist of a skirt 14 substantially flat capping the upper part of the stent 4 of a prosthesis according to the invention. The underside of the skirt 14—the one intended to face the left ventricle of a heart during implantation of the prosthesis, has spikes or hooks 15 which the respective distal parts are advantageously harpoons. The opposite face of said skirt 14 remains present in the left atrium above the annulus of the native mitral valve. The invention provides that the upper side of the skirt is not necessarily plan. The upper part may possibly thus be curved. The invention provides that the skirt 14 may fit closely the morphology of the annulus of a native mitral valve. This skirt is then annular. The anchoring of the prosthesis is thus optimized. Alternatively, as described in the example shown in FIG. 4C, the skirt may fit only the upper part of the stent 4. Such a skirt has a shape (top view) like a “C” in order to fit closely the cap of a stent which the configuration would be close to a vertical half-cylinder.

A skirt 14 (whatever its form) allows to seal the mitral valve prosthesis on the annulus of a native mitral valve during implantation of the prosthesis. It also allows to perfectly adjust the shape of the valve prosthesis to the one of the annulus—for example using a balloon. The skirt 14 may also prevent paravalvular leakage. Such a skirt 14 may thus complement or alternatively the sealing membrane 10 discussed in conjunction with FIG. 2.

A mitral valve prosthesis according to the invention may be deployed at the level of the annulus of a native mitral valve from different accesses, such as the apex of the left ventricle (transapical access), the femoral vein (transvenous-transseptal access), the jugular vein (transseptal access), the subclavian vein (transseptal access) or the right upper pulmonary vein.

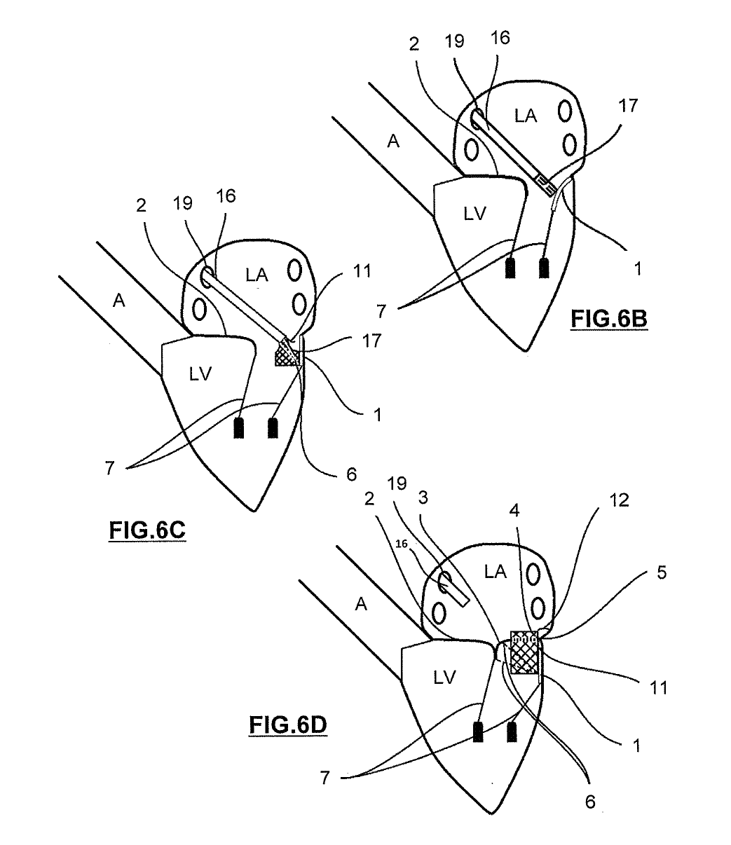

FIGS. 5A to 5C, each show a sectional view of a heart on which is represented a delivery catheter 16 having and carrying a prosthetic mitral valve 17 according to the present invention. Said figures respectively describe three main steps of a method for implanting via a catheter and an apical approach a prosthesis according to the invention and according to the embodiment described in connection with FIG. 2. According to FIG. 5A, the implantation process is first to carry the prosthesis 17 at the level of the annulus of the native mitral valve from an apical approach (i.e. direct access to the mitral valve through the apex 9 of the left ventricle LV). The delivery catheter 16 passes through the apex 9 of the left ventricle, from a mini-thoracotomy of a few centimeters, progresses within the left ventricle LV into the left atrium LA through the native mitral valve.

FIG. 5B describes the initial deployment of the mitral valve prosthesis 17 mainly in the left atrium LA. The leaflet 3 of the mitral valve prosthesis is almost in position, while the upper part of the stent 4 and the cords 6 are still being deployed. A series of hooks 12 used for anchoring the mitral valve prosthesis 17 into the left atrium LA is attached to the wall of said atrium. A second set of hooks 11 used for anchoring of the prosthetic mitral valve 17 into the left ventricle LV has not yet penetrated the tissues of the inner wall of the left ventricle.

FIG. 5C shows the prosthetic mitral valve 17 in nominal position and fully deployed. The delivery catheter 16 can then be removed of the left ventricle LV from its apex 9.

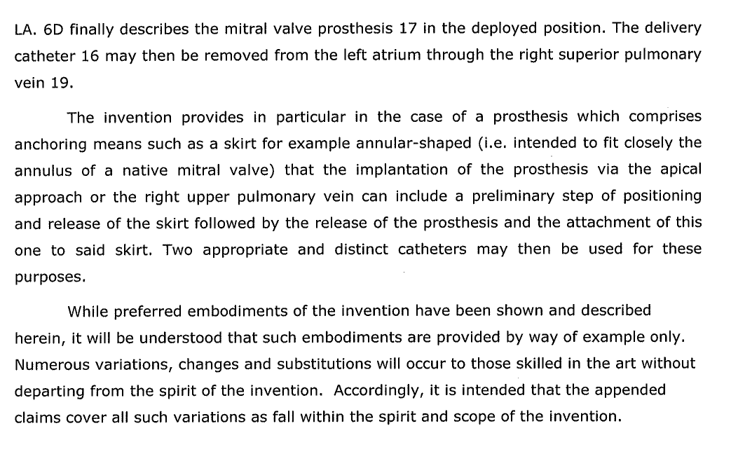

FIGS. 6A to 6D respectively describe four stages of a second method for implanting via a catheter an equivalent prosthesis according to the invention. The implantation is performed by accessing a heart from the right superior pulmonary vein 19.

According to FIG. 6A, a mitral valve prosthesis 17 is led at the level of the annulus of the native mitral valve by a delivery catheter 16 from the upper right pulmonary vein 19 (direct access to the mitral valve through the left atrium). The delivery catheter 16 having the prosthesis 17 leaves the right upper pulmonary vein 19 and gets in the left atrium LA. As shown in FIG. 6B, the delivery catheter 16 progresses through the native mitral valve and into the left ventricle LV. According to 6C, the deployment of the mitral valve prosthesis 17 starts since mainly in the left ventricle LV. The lower part of the stent 4 and the cords 6 of the prosthesis are partially deployed. The leaflet 3 of the prosthesis is not yet in its nominal position. A first set of hooks 11 used for anchoring the mitral valve prosthesis 17 into the left ventricle LV is deployed but has not yet penetrated the tissues of the inner wall of the left ventricle. A second set of hooks 12 used for anchoring the prosthesis 17 in the left atrium LA has not yet been deployed. At this stage of the implantation process, the upper part of the stent 4 of the prosthesis is not yet attached to the wall of the left atrium

1. A mitral valve prosthesis comprises:
a docking station and
a deflectable mono-leaflet comprising a membrane, said leaflet cooperating with said docking station by attachment means and being arranged in a configuration near a posterior leaflet of a native mitral valve (MV), said mitral valve prosthesis configured to be implantable into the native mitral valve using a catheter.

2. The prosthesis according to claim 1, wherein the leaflet membrane is made from one or several biological materials.

3. The prosthesis according to claim 1, wherein the leaflet membrane is made from one or more synthetic materials.

4. The prosthesis of claim 1, wherein the leaflet is substantially flat and the attachment means compel the proximal part and the sides of the leaflet united of the docking station.

5. The prosthesis according to claim 1, wherein the leaflet is substantially flat and the attachment means compel the proximal part and partially the sides of the leaflet united of the docking station, the distal part of aforesaid leaflet remaining free with respect to the proximal part of said leaflet.

6. The prosthesis according to claim 5, further comprising holding means co-operating firstly with the free part of the leaflet and secondly with the docking station.

7. The prosthesis of claim 5, further comprising holding means arranged to cooperate with, firstly the free part of the leaflet and secondly after implantation of the prosthesis within the native mitral valve (MV) of a patient with a fibrous head of native papillary muscles or the left ventricle (LV) of the patient's heart.

8. The prosthesis according to claim 6, wherein the holding means consist of one or more cords.

9. The prosthesis according to claim 8, wherein the one or more cords are made of xenograft of animal pericardium coated with glutaraldehyde.

10. The prosthesis of claim 8, wherein the cords are synthetic.

11. The prosthesis of claim 5, wherein the leaflet is made from a shape memory material.

12. The prosthesis according to claim 1, wherein the attachment means to ensure cooperation between the docking station and the leaflet consist of stitches, eyelets, staples or clips.

13. The prosthesis according to claim 1, including anchoring means for anchoring of the prosthesis after implantation.

14. The prosthesis according to claim 13, wherein the anchoring means consist of hooks, clamps or spikes.

15. The prosthesis of claim 13 wherein the anchoring means consist of a projecting skirt capping an upper part of the docking station and whose bottom face intended to face in a left ventricle during the prosthesis implantation, includes one or more spikes or hooks.

16. The prosthesis of claim 1, wherein the docking station comprises a substantially tubular stent whose shape is substantially that of a half-cylinder and wherein the proximal part of the leaflet fits into said stent in a substantially orthogonal plane to the axis of revolution of the stent.

17. The prosthesis according to claim 16, wherein the stent consists of a mesh of one or more wires of Nickel-Titanium or Nitinol, stainless steel, chrome-cobalt, or titanium.

18. The prosthesis of claim 1, wherein the leaflet comprises a sealing membrane.

19. The prosthesis according to claim 18, wherein the sealing membrane is attached to the docking station by fixing means.

20. The prosthesis according to claim 18, wherein the sealing membrane is made from animal pericardium or synthetic materials.

21. A delivery system having a mitral valve prosthesis comprising:
a docking station and
a deflectable mono-leaflet comprising a membrane, said leaflet cooperating with said docking station by attachment means and being arranged in a configuration near a posterior leaflet of a native mitral valve (MV), said mitral valve prosthesis configured to be implantable into the native mitral valve using a catheter.

22. The prosthesis according to claim 1, wherein the leaflet is replaceable while the docking station remains implanted.
